(19) United States
(12) Patent Application Publication
(10) Pub. No.: US 2011/0070294 A1
(43) Pub. Date: Mar. 24, 2011

(54) Methods for the Administration of Drugs Using Liposomes

(76) Inventors: Indu Javeri, Andover, MA (US); Kaliappanadar Nellaiappan, Lexington, MA (US); Bharat Dixit, Hingham, MA (US)

(21) Appl. No.: 12/889,272

(22) Filed: Sep. 23, 2010

Related U.S. Application Data
(60) Provisional application No. 61/245,234, filed on Sep. 23, 2009.

(51) Int. Cl. A61K 9/127; A61K 31/337; A61K 31/352; A61K 38/13; A61K 38/40; A61P 35/00; A61P 37/08; A61P 7/06

(52) U.S. Cl. 424/450; 514/449; 514/454; 514/20.5; 514/13.5

(57) ABSTRACT

Provided herein are methods for the administration of drugs using liposomes. In particular, provided herein is the use of liposomes as diluents for lyophilized or liquid drugs to deliver the drug safely, reduce the toxicity of the drug or the excipients used to solubilize or stabilize the drug.

### CROSS REFERENCE TO RELATED APPLICATIONS

This application claims the benefit under 35 U.S.C. §119 of U.S. Provisional Patent Appl. No. 61/245,234, filed Sep. 23, 2009, the disclosure of which is incorporated by reference in its entirety herein.

Provided herein are methods for the administration of drugs using liposomes. In particular, provided herein is the use of liposomes as diluents for lyophilized or liquid drugs to deliver the drug safely, reduce the toxicity of the drug or the excipients used to solubilize or stabilize the drug.

### BACKGROUND

The bioavailability of a pharmaceutical drug depends largely in part on the solubility and stability of the drug. The dissolution of drugs in a pharmaceutically acceptable medium is the most preferred manner of formulation to deliver a drug, and many drug candidates do not reach the market due to their poor solubility. Many attempts have been made to improve the solubility of drugs having poor solubility. These approaches include: reduction of particle size of a compound using techniques such as micronization and nanosuspensions; modification of the crystal habit of a compound; complexation with other agents such as cyclodextrins; dispersion of a compound in a highly soluble carrier; solubilization in organic solvent; associating the drug in micelles of detergents; or encapsulating the drug in a liposome bilayer or liposome. See e.g., U.S. Pat. No. 7,345,093; Strickley, R. G., Pharmaceutical Research, vol. 21, pp. 201-230 (2004); Pinnamaneni et al., Pharmazie, vol. 57, no. 5, pp. 291-300 (2002).

Either the drug itself or the excipients used to solubilize the drug may have side effects such as allergic reaction or hemolysis. Most of the commonly used carrier agents, in particular polyoxyethylene sorbitan monooleate (i.e. polysorbates) and CREMOPHOR® EL, exhibit various clinical side effects, especially in children and newborns. For example, the currently approved formulation of paclitaxel, a commonly prescribed anti-cancer agent, contains 51% CREMOPHOR® EL and 49% ethanol. CREMOPHOR® EL is believed to cause hypersensitive reactions to paclitaxel treatments. In addition, CREMOPHOR® EL has been reported to decrease the bioavailability of paclitaxel. See Baedelmeijer et al., Cancer Chemother. Pharmacol, vol. 49, pp. 119-125 (2002). This decrease in bioavailability necessitates the administration of higher doses of the paclitaxel formulation, which, in turn, results in a higher risk of hypersensitive reactions to the paclitaxel formulation. Moreover, in drug formulations, such as paclitaxel, in which ethanol is present in the formulation and of which high doses are required, acute ethanol toxicity is a concern.

Organic solvents are often used in injectable pharmaceutical solutions, for example, as a carrier or diluent. However, many organic solvents, for example, ethanol, propylene glycol, polyethylene glycol, dimethylacetamide, and dimethylsulfoxide, are hemolytic and thus are undesirable for use in injectable solutions. Other limitations in using organic solvents in injectable products include precipitation, pain, and inflammation upon injection. Moreover, liposomal formulations used to improve the solubility and bioavailabilities of a drug have limited compatibility with organic solvents, as liposomes are not stable in concentrated organic solvent-containing formulations.

To reduce these side effects, attempts have been made by taking premedication to reduce allergic reactions. The use of routine aqueous diluents such as 5% D5W (dextrose 5%) or sodium chloride will not prevent side effects such as hemolysis, however, and aqueous diluents alone cannot often be used due to the poor water solubility of certain drugs. Thus there is a need for diluents compatible with compositions comprising hydrophobic drugs and/or drugs with poor aqueous solubility without the use of organic solvents or other harmful diluents.

Lipid emulsions with high amounts of oil (e.g., 20% by weight) diminish the hemolytic properties of hemolysin by reducing the concentration of hemolysin through binding. See, e.g., Jumaa & Muller, European Journal of Pharmaceutical Sciences, 9 (2000) 285-290). Various side effects of oil emulsions are well documented. Moreover, a lipid oil emulsion itself will have hemolytic properties. See, e.g., Jumaa et al., Pharmaceutica Acta Helvetiae, vol. 73, issue 6, pp. 293-301 (1999).

Liposomes are microscopic lipid vesicles that are composed of a central aqueous cavity surrounded by a lipid membrane formed by concentric bilayer(s) (lamellas). Liposomes are able to incorporate hydrophilic substances (in the aqueous interior) or hydrophobic substances (in the lipid membrane). Liposomes can be unilamellar vesicles (“UMV”) having a single lipid bilayer, or multilamellar vesicles (“MLV”), having a series of lipid bilayers (also referred to as “oligolamellar vesicles”). The multilamellar vesicles typically range in size from 0.2 μm to 10 μm in diameter. Unilamellar vesicles with a diameter of less than 0.2 μm (e.g. between 0.02 μm and 0.2 μm) are commonly known as small unilamellar vesicles (“SUV”). See e.g., WO 98/006882. Unilamellar vesicles with a diameter greater than 0.45 μm (in some cases greater than 1 μm) are commonly known as large unilamellar vesicles (“LUV”).

The bilayer(s) of liposomes most often comprise phospholipids, but may also comprise lipids including but not limited to fatty acids, fatty acid salts and/or fatty alcohols. The properties of the liposomes depend, among other factors, on the nature of the constituents. Consequently, if liposomes with certain characteristics are to be obtained, the charge of its polar group and/or the length and the degree of saturation of its fatty acid chain must be taken into account.

There are numerous options for combining phospholipids, optionally with other lipids or cholesterol, with an aqueous phase to obtain liposomes. Depending on the method of preparation and the lipids used, it is possible to obtain vesicles of different sizes, structures and properties. Methods for the preparation of liposomes and liposomal solutions are readily available in the art. See, e.g., Bangham et al., J. Mol. Biol., vol. 11, pp. 238-252 (1965); Szoka and Papandjopoulos, Ann. Rev. Biophys. Bioeng., vol. 2, pp. 467-508 (1980); Dousset and Douste-Blazy, Les Liposomes. Puisieux and Delattre, Editors, Tecniques et Documentation Lavoisier, Paris, pp. 1-73 (1985); WO 92/10166; WO 02/080883; U.S. Pat. No. 4,508,703; and U.S. Pat. App. Pub. No. 2008/0274172.

Consequently, there exists a need for additional and/or improved diluents or methods of administering drugs using diluents, particularly for the parenteral administration of drugs with poor solubility. There is also a need for diluents and methods that reduce the side effects associated with the administration of lyophilized or aqueous drugs.

### SUMMARY

Provided herein are methods for the administration of drugs using liposomes.

Also provided herein is the use of liposomes as diluents for pharmaceutical formulations to deliver the drug safely, reduce the toxicity of the drug or the excipients used to solubilize or stabilize the drug.

In one embodiment, provided herein is the use of an aqueous liposomal solution as a diluent for a lyophilized or liquid drug formulation.

The encapsulation of drugs into liposomes is well-known in the art. Provided herein are methods in which little or no encapsulation occurs when a solution of liposomes is added to a pharmaceutical formulation or otherwise used as a diluent in a pharmaceutical formulation. As used herein, “little encapsulation” refers to any incidental encapsulation that may occur by mixing a solution of the liposomes provided herein with a composition or formulation comprising one or more drugs (i.e., less than about 10%, 5%, 2%, 1% or 0.1% encapsulation).

In one embodiment, provided herein is a method for the administration of drugs using liposomes, wherein the liposomes used as a diluent do not encapsulate the drug or drugs into the liposomes.

In certain embodiments, the drug is hydrophobic or has poor aqueous solubility.

In one embodiment, the lyophilized or liquid drug formulation is suitable for parenteral administration to a patient.

The methods provided herein may be used in the treatment of diseases or disorders. In one embodiment, the liposomal preparation is suitable for parenteral administration to a patient suffering from said disease or disorder. In one embodiment, the patient is a human.

### DEFINITIONS

As used herein, and unless otherwise specified, “lipid” is understood to be a fatty acid, fatty acid salt, fatty alcohol, or phospholipid. Lipids may also include sterols, including but not limited to cholesterol; sphingolipids, including but not limited to sphingomyelin; glycosphingolipids including but not limited to gangliosides, globocides and cerebrosides; and surfactant amines including but not limited to stearyl, oleyl and linoleyl amines.

As used herein, and unless otherwise specified, “phospholipid” is understood to be an amphyphilic derivative of glycerol in which one of its hydroxyl groups is esterified with phosphoric acid and the other two hydroxyl groups are esterified with long-chain fatty acids which can be equal to or different from each other and can be saturated or unsaturated. A neutral phospholipid is generally one in which the other phosphoric acid hydroxyl is esterified by an alcohol substituted by a polar group (usually hydroxyl or amino) and whose net charge is zero. A phospholipid with a charge is generally one in which the other phosphoric acid hydroxyl is esterified by an alcohol substituted by a polar group and whose net charge is positive or negative.

Examples of phospholipids include, but are not limited to phosphatidic acid (“PA”), phosphatidylcholine (“PC”), phosphatidylglycerol (“PG”), phophatidylethanolamine (“PE”), phophatidylinositol (“PI”), and phosphatidylserine (“PS”), sphingomyelin (including brain sphingomyelin), lecithin, lysolecithin, lysophosphatidylethanolamine, cerebrosides, diarachidoylphosphatidylcholine (“DAPC”), didecanoyl-L-alpha-phosphatidylcholine (“DDPC”), dielaidoylphosphatidylcholine (“DEPC”), dilauroylphosphatidylcholine (“DLPC”), dilinoleoylphosphatidylcholine, dimyristoylphosphatidylcholine (“DMPC”), dioleoylphosphatidylcholine (“DOPC”), dipalmitoylphosphatidylcholine (“DPPC”), distearoylphosphatidylcholine (“DSPC”), 1-palmitoyl-2-oleoyl-phosphatidylcholine (“POPC”), diarachidoylphosphatidylglycerol (“DAPG”), didecanoyl-L-alpha-phosphatidylglycerol (“DDPG”), dielaidoylphosphatidylglycerol (“DEPG”), dilauroylphosphatidylglycerol (“DLPG”), dilinoleoylphosphatidylglycerol, dimyristoylphosphatidylglycerol (“DMPG”), dioleoylphosphatidylglycerol (“DOPG”), dipalmitoylphosphatidylglycerol (“DPPG”), distearoylphosphatidylglycerol (“DSPG”), 1-palmitoyl-2-oleoyl-phosphatidylglycerol (“POPG”), diarachidoylphosphatidylethanolamine (“DAPE”), didecanoyl-L-alpha-phosphatidylethanolamine (“DDPE”), dielaidoylphosphatidylethanolamine (“DEPE”), dilauroylphosphatidylethanolamine (“DLPE”), dilinoleoylphosphatidylethanolamine, dimyristoylphosphatidylethanolamine (“DMPE”), dioleoylphosphatidylethanolamine (“DOPE”), dipalmitoylphosphatidylethanolamine (“DPPE”), distearoylphosphatidylethanolamine (“DSPE”), 1-palmitoyl-2-oleoyl-phosphatidylethanolamine (“POPE”), diarachidoylphosphatidylinositol (“DAPI”), didecanoyl-L-alpha-phosphatidylinositol (“DDPI”), dielaidoylphosphatidylinositol (“DEPI”), dilauroylphosphatidylinositol (“DLPI”), dilinoleoylphosphatidylinositol, dimyristoylphosphatidylinositol (“DMPI”), dioleoylphosphatidylinositol (“DOPI”), dipalmitoylphosphatidylinositol (“DPPI”), distearoylphosphatidylinositol (“DSPI”), 1-palmitoyl-2-oleoyl-phosphatidylinositol (“POPI”), diarachidoylphosphatidylserine (“DAPS”), didecanoyl-L-alpha-phosphatidylserine (“DDPS”), dielaidoylphosphatidylserine (“DEPS”), dilauroylphosphatidylserine (“DLPS”), dilinoleoylphosphatidylserine, dimyristoylphosphatidylserine (“DMPS”), dioleoylphosphatidylserine (“DOPS”), dipalmitoylphosphatidylserine (“DPPS”), distearoylphosphatidylserine (“DSPS”), 1-palmitoyl-2-oleoyl-phosphatidylserine (“POPS”), diarachidoyl sphingomyelin, didecanoyl sphingomyelin, dielaidoyl sphingomyelin, dilauroyl sphingomyelin, dilinoleoyl sphingomyelin, dimyristoyl sphingomyelin, sphingomyelin, dioleoyl sphingomyelin, dipalmitoyl sphingomyelin, distearoyl sphingomyelin, and 1-palmitoyl-2-oleoyl-sphingomyelin.

As used herein, and unless otherwise specified, the term “hydrophobic compound” means a compound with little or no water solubility. In some embodiments, a hydrophobic compound has an intrinsic water solubility (i.e., water solubility of the unionized form) of less than about 20 percent by weight, about 15 percent by weight, about 10 percent by weight, about 5 percent by weight, about 1 percent by weight, about 0.1 percent by weight or about 0.01 percent by weight. In other embodiments, a hydrophobic compound has an intrinsic water solubility of less than about 10 mg/mL, about 7 mg/mL, about 5 mg/mL, about 3 mg/mL, about 1 mg/mL or about 0.1 mg/mL.

As used herein, or unless otherwise specified, the terms “aqueous medium” and “aqueous solution” mean any water based medium or solution, e.g., water, a saline solution, a sugar solution, a transfusion solution, a buffer, and any other readily available water-based medium. Further, an aqueous medium or solution may contain one or more water soluble organic solvents. In the case of a parenteral solution, an aqueous medium is preferably sterile and suitable for use as a carrier of an active agent. Examples of an aqueous medium include, but are not limited to, water for injection, saline solution, Ringer's solution, D5W or other solutions of water-miscible substances such as dextrose and other electrolytes.

As used herein, and unless otherwise specified, “encapsulate” or “encapsulation” is understood to be the process of incorporating an active agent or drug into liposomes or liposomal vesicles. The encapsulated active agent can remain in the aqueous interior or associate with membranes.

As used herein in connection with liposomes or liposomal solutions and preparations, and unless otherwise specified, “diluent” or “used a diluent” means that little or no encapsulation of an active agent or drug occurs.

As used herein, “little encapsulation” refers to any incidental encapsulation that may occur by mixing a solution of liposomes provided herein with a composition or formulation comprising one or more drugs.

As used herein, and unless otherwise specified, the term “fatty acid” means a compound whose structure is a carboxylic group attached to a hydrocarbon chain having one or more carbon atoms. The hydrocarbon chain may be saturated or unsaturated (i.e., alkyl, alkenyl or alkynyl hydrocarbon chains). Also, the hydrocarbon chain may be straight or branched. Moreover, in some embodiments, hydrogens in the hydrocarbon chain may be substituted.

As used herein, and unless otherwise specified, the term “fatty alcohol” means a compound whose structure is an alcohol group attached to a hydrocarbon chain having one or more carbon atoms. The hydrocarbon chain may be saturated or unsaturated (i.e., alkyl, alkenyl or alkynyl hydrocarbon chains). Also, the hydrocarbon chain may be straight or branched. Moreover, in some embodiments, hydrogens in the hydrocarbon chain may be substituted.

As used herein, and unless otherwise specified, the term “fatty acid salt” means a compound formed from a reaction between a fatty acid and an inorganic/organic base. In addition, the term also encompasses a compound formed from a reaction between a fatty alcohol and an inorganic/organic acid. Examples of such acids include, but are not limited to, sulfuric and phosphoric acid. The hydrocarbon chain of the fatty acid salt may be saturated or unsaturated (i.e., alkyl, alkenyl or alkynyl hydrocarbon chains). In addition, the hydrocarbon chain may be straight or branched. Moreover, in some embodiments, hydrogens in the hydrocarbon chain may be substituted.

As used herein, and unless otherwise specified, the term “substituted” means a group substituted by one or more substituents including, but not limited to, alkyl, alkenyl, alkynyl, cycloalkyl, aroyl, halo, haloalkyl (e.g., trifluoromethyl), substituted or unsubstituted heterocycloalkyl, haloalkoxy (e.g., trifluoromethoxy), hydroxy, alkoxy, cycloalkyloxy, heterocylooxy, oxo, alkanoyl, aryl, substituted aryl, substituted or unsubstituted heteroaryl (e.g., indolyl, imidazolyl, furyl, thienyl, thiazolyl, pyrrolidyl, pyridyl, pyrimidyl and the like), arylalkyl, alkylaryl, heteroaryl, heteroarylalkyl, alkylheteroaryl, heterocyclo, aryloxy, alkanoyloxy, amino, alkylamino, arylamino, arylalkylamino, cycloalkylamino, heterocycloamino, mono- and di-substituted amino, alkanoylamino, aroylamino, aralkanoylamino, substituted alkanoylamino, substituted arylamino, substituted aralkanoylamino, carbamyl (e.g., CONH₂), substituted carbamyl (e.g., CONH-alkyl, CONH-aryl, CONH-arylalkyl or instances where there are two substituents on the nitrogen), carbonyl, alkoxycarbonyl, carboxy, cyano, ester, ether, guanidino, nitro, sulfonyl, alkylsulfonyl, arylsulfonyl, arylalkylsulfonyl, sulfonamido (e.g., SO₂NH₂), substituted sulfonamido, thiol, alkylthio, arylthio, arylalkylthio, cycloalkylthio, heterocyclothio, alkylthiono, arylthiono and arylalkylthiono.

As used herein, and unless otherwise specified, the term “alkyl” means a saturated straight chain or branched non-cyclic hydrocarbon having from 1 to 20 carbon atoms, preferably 1-10 carbon atoms and most preferably 1-4 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl; while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. An alkyl group can be unsubstituted or substituted. Unsaturated alkyl groups include alkenyl groups and alkynyl groups, which are discussed below.

As used herein, and unless otherwise specified, the term “alkenyl” means a straight chain or branched non-cyclic hydrocarbon having from 2 to 20 carbon atoms, more preferably 2-10 carbon atoms, most preferably 2-6 carbon atoms, and including at least one carbon-carbon double bond. Representative straight chain and branched (C₂-C₁₀) alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like. The double bond of an alkenyl group can be unconjugated or conjugated to another unsaturated group. An alkenyl group can be unsubstituted or substituted.

As used herein, and unless otherwise specified, the term “alkynyl” means a straight chain or branched non-cyclic hydrocarbon having from 2 to 20 carbon atoms, more preferably 2-10 carbon atoms, most preferably 2-6 carbon atoms, and including at lease one carbon-carbon triple bond. Representative straight chain and branched (C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl, and the like. The triple bond of an alkynyl group can be unconjugated or conjugated to another unsaturated group. An alkynyl group can be unsubstituted or substituted.

As used herein, and unless otherwise specified, the term “pharmaceutically acceptable salt” refers to a salt prepared from pharmaceutically acceptable non-toxic acids or bases including inorganic acids and bases and organic acids and bases. Suitable pharmaceutically acceptable base addition salts for the compositions provided herein include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} ed., Mack Publishing, Easton Pa. (1990) or Remington: The Science and Practice of Pharmacy, 19th ed., Mack Publishing, Easton Pa. (1995).

As used herein, the term “hydrate” means a compound provided herein, or a salt thereof, that further includes a stoichiometric or non-stoichiometric amount of water bound by non-covalent intermolecular forces.

As used herein, the term “clathrate” means a compound provided herein, or a salt thereof in the form of a crystal lattice that contains spaces (e.g., channels) that have a guest molecule (e.g., a solvent or water) trapped within.

As used herein, and unless otherwise indicated, the term “prodrug” means a derivative of a compound that can hydrolyze, oxidize, or otherwise react under biological conditions (in vitro or in vivo) to provide an active compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. Esterifying any of the carboxylic acid moieties present on the molecule conveniently forms the carboxylate esters. Prodrugs can typically be prepared using well-known methods, such as those described by Burger's Medicinal Chemistry and Drug Discovery 6th ed. (Donald J. Abraham ed., 2001, Wiley), and Design and Application of Prodrugs (H. Bundgaard ed., 1985, Harwood Academic Publishers Gmfh).

As used herein, and unless otherwise specified, the term “stable,” when used in connection with a formulation, means that the active agent of the formulation, when prepared using the methods provided herein, remains solubilized for a specified amount of time and does not significantly degrade or aggregate or become otherwise modified (as determined, for example, by HPLC).

As used herein, and unless otherwise specified, the term “harmful ingredient,” when used in connection with pharmaceutical compositions, means an ingredient commonly used in a pharmaceutical composition that may cause clinical side effects such as, but not limited to, hemolysis, hypersensitive reaction, peripheral neuropathies, and/or a decrease in the bioavailability of the active ingredient of the composition. Examples of harmful ingredients include, but are not limited to: toxic solvents, including organic solvents such as ethanol, methanol, 1-propanol, 2-propanol, acetone, acetonitrile, ethyl acetate, methyl acetate, diethyl ether, dimethyl ether, diisopropyl ether, methyl tert-butyl ether (“MTBE”), tetrahydrofuran (“THE”), dichloromethane, chloroform, carbon tetrachloride, 1,2-dicholroethane, pentane, hexanes, heptane, petroleum ether, dioxane, ethylene glycol, diethylene glycol, diglyme, 1,2-dimethoxyethane, 1-butanol, 2-butanol, 2-butanone, benzene, toluene, dimethylsulfoxide (“DMSO”), dimethylformamide (“DMF”), hexamethylphosphoramide (“HMPA”), N-methylpyrrolidone, glycerin, nitromethane, triethyl amine, xylenes, CREMOPHOR® EL, and polyethylene glycol (“PEG”); co-detergents or surfactants such as poly sorbates or vitamin E; oils such as Castor oil or corn oil; proteins such as HSA; or any other biologic which is potential source of contamination.

### FIGURES

FIG. 1 depicts the reduction of hemolysis associated with compositions reconstituted with liposomes.

FIG. 2 depicts the maximum tolerant dose for Miradocetaxel in mice.

### DETAILED DESCRIPTION

Provided herein are methods for the administration of drugs using liposomes.

Also provided herein is the use of liposomes as diluents for pharmaceutical formulations to deliver the drug safely, reduce the toxicity of the drug or the excipients used to solubilize or stabilize the drug.

In one embodiment, provided herein is the use of an aqueous liposomal solution as a diluent for a lyophilized or liquid drug formulation.

In one embodiment, provided herein is a method for the administration of drugs using liposomes, wherein the liposomes used as a diluent do not encapsulate the drug or drugs into the liposomes. In certain embodiments, little to no encapsulation of the drug occurs, i.e., any encapsulation that may occur is merely incidental, resulting from the mixture a solution of liposomes with a drug formulation as provided herein.

Liposomes and solutions of liposomes may be prepared by any of those methods known in the art. Examples of such methods include, but are not limited to those taught in: U.S. Provisional Pat. App. No. 61/245,185 (“METHODS FOR THE PREPARATION OF LIPOSOMES”); U.S. Pat. App. Pub. No. 2008/0274172; U.S. Pat. App. Pub. No. 2008/0102109; U.S. Pat. App. Pub. No. 2007/0631460; U.S. Pat. No. 7,345,093; U.S. Pat. No. 4,508,703; WO 92/10166; Bangham et al., J. Mol. Biol., 11:238-252 (1965); Szoka and Papandjopoulos, Ann. Rev. Biophys. Bioeng., 2: 467-508 (1980), and Dousset and Douste-Blazy, Les Liposomes, Puisieux and Delattre, Editors, Tecniques et Documentation Lavoisier, Paris, pp. 1-73 (1985).

In certain embodiments, the drug is hydrophobic or has poor aqueous solubility.

In other embodiments, the drug is not hydrophobic.

In one embodiment, the lyophilized or liquid drug formulation is suitable for parenteral administration to a patient.

In one embodiment, the methods provided herein may be used in the treatment of diseases or disorders. In one embodiment, the liposomal preparation is suitable for parenteral administration to a patient suffering from said disease or disorder.

In one embodiment, the patient is a human.

In one embodiment, the drugs used in the methods provided herein, or the pharmaceutically acceptable salts, hydrates, clathrates or prodrugs thereof, include, but are not limited to, β-lapachone, taxanes (including but not limited to taxol, 7-epitaxol, 7-acetyl taxol, 10-desacetyltaxol, 10-desacetyl-7-epitaxol, 7-xylosyltaxol, 10-desacetyl-7-sylosyltaxol, 7-glutaryltaxol, 7-N,N-dimethylglycycltaxol, 7-L-alanyltaxol, taxotere, and mixtures thereof), docetaxel, paclitaxel, colchicine, transferrin, cyclosporines, cyclosporin A, ketoprofen, propofol, acetylsalicylic acid, acetaminophen, amphotericin, digoxin, doxorubicin, daunorubicin, epirubicin, idarubicin, angiogenesis inhibitors (e.g., bevacizumab, ranibizumab, vitaxin, carboxyamidotriazole, combretastatin A-4, fumagillin analogs (e.g., TNP-470), CM101, IFN-α, interleukin-10, interleukin-12, platelet factor-4, suramin, SU5416, thrombospondin, VEGFR antagonists, angiostatin, endostatin, 2-methoxyestradiol, tecogalan, thalidomide, prolactin, linomide, angiopoietin-1, basic fibroblast growth factor, vascular endothelial growth factor), vinca-alkaloids (e.g., vinblastine, vincristine, vindesin, etoposide, etoposide phosphate, and teniposide), cytarabine, actinomycin, etoposide, bleomycin, gentamycin, cyclophosphamide, methotrexate, streptozotocin, cytosine, β-D-arabinofuranoside-5′-triphosphate, cytochrome C, cisplatin, N-phosphono-acetyl-L-aspartic acid, 5-fluoroorotic acid, acyclovir, zidovudine, interferons, aminoglycosides, cephalosporins, tetracyclines, propranolol, timolol, labetolol, clonidine, hydralazine, imipramine, amitriptyline, doxepim, phenyloin, diphenhydramine, chlorphenirimine, promethazine, prostaglandins, methotrexate, progesterone, testosterone, estradiol, estrogen, epirubicin, beclomethasone and esters, vitamin E, cortisone, dexamethasone and esters, betamethasone valerete, biphenyl dimethyl dicarboxylic acid, calcitonins, camptothecin, captopril, cephazoline, chloroquinine, chlorothiazole, co-agulation factors VIII and IX, d-alpha-tocopherol, dexamethasone, dichlofenac, etoposide, feldene, flubiprofen, 5-fluorouracil, fluoxetine, fusidic acid, gentamicin, glyburide, granisetron, growth hormones, indomethacin, insulin, itraconazole, ketoconazole, methotrexate, metronidazole, minoxidil, mitomycin, nafcillin, naproxen, ondansetron, oxyphenbutazone, parazosin, physostigmine, piroxicam, prednisolone, primaquine, quinine, ramipril, taxotane, tenoxicam, terazosin, triamcinolone, urokinase, opioid analgesics (e.g., alfentanil, anileridine, codiene, diamorphine, fentanyl, hydrocodone, hydromorphone, meperidine, morphine, oxycodone, oxymorphone, propoxyphene, sufentanil, pentazocine and nalbuphine), non-steroidal anti-inflammatory drugs (e.g., aspirin, indometacin, ibuprofen, mefenamic acid and phenylbutazone), angiotensin converting enzyme (“ACE”) inhibitors (e.g., captoprilpolyene), protein kinase C inhibitors, antibiotics (e.g., imidazole and triazole antibiotics), folic acid, anthracycline antibiotics, anti-sense RNAs, tricathecums, microbial ribosomal-inactivating toxins (e.g., gelonin, abrin, ricin A chain, Pseudomonas exotoxin, diptheria toxin, pokeweed antiviral peptide), pipecolic acid derivatives (e.g., tacrolimus), plant alkaloids, dyes, radioisotope-labeled compounds, radiopaque compounds, radiosensitizers (e.g., 5-chloro-2′-deoxyuridine, 5-bromo-2′-deoxyuridine and 5-iodo-2′-deoxyuridine), fluorescent compounds, mydriatic compounds, bronchodilators, local anesthetics (e.g., dibucaine and chlorpromazine), antifungal agents (e.g., miconazole, terconazole, econazole, isoconazole, butaconazole, clotrimazole, itraconazole, nystatin, naftifine and amphotericin B), antiparasitic agents, hormones, hormone antagonists, immunomodulators, neurotransmitter antagonists, anti-diabetic agents, antiglaucoma agents, vitamins, narcotics, and imaging agents. For additional disclosure of active agents, see Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W.B. Saunders, Philadelphia, 1996.

In some embodiments, the methods provided herein eliminate or reduce the need for toxic solvents such as CREMOPHOR® EL, N-methylpyrrolidone, dimethylformamide, and DMSO in the formulation. Furthermore, co-solvents such as ethanol or polyethylene glycol (“PEG”), co-detergents or co-surfactants such as polysorbates (e.g., Tweens) or vitamin E, oils such as Castor oil or corn oil, and proteins such as HSA are not necessary for the dilution of a composition according to the methods provided herein.

Therefore, in one embodiment, the methods provided herein eliminate the need for the use of solvents including, but not limited to, ethanol, methanol, 1-propanol, 2-propanol, acetone, acetonitrile, ethyl acetate, methyl acetate, diethyl ether, dimethyl ether, diisopropyl ether, methyl tert-butyl ether (“MTBE”), tetrahydrofuran (“THF”), dichloromethane, chloroform, carbon tetrachloride, 1,2-dicholroethane, pentane, hexanes, heptane, petroleum ether, dioxane, ethylene glycol, diethylene glycol, diglyme, 1,2-dimethoxyethane, 1-butanol, 2-butanol, 2-butanone, benzene, toluene, dimethylsulfoxide (“DMSO”), dimethylformamide (“DMF”), hexamethylphosphoramide (“HMPA”), N-methylpyrrolidone, glycerin, nitromethane, triethyl amine, xylenes, CREMOPHOR® EL, and polyethylene glycol (“PEG”).

In another embodiment, the methods provided herein eliminate the need for the use of co-detergents or surfactants such as polysorbates or vitamin E, oils such as Castor oil or corn oil, proteins such as HSA, or any other biologic which is potential source of contamination.

The pharmaceutical compositions provided herein may further comprise one or more excipients. The compositions may be prepared suitable for any route of administration, including, but not limited to, oral, mucosal (such as nasal or pulmonary), topical, transdermal and parenteral administration.

In one embodiment, provided herein is a method of in vivo delivery of one or drugs, the drug being previously formulated for parenteral delivery, comprising adding a solution of liposomes to the formulation. In certain embodiments, the previously formulated drugs are lyophilized. In other embodiments, the previously formulated drugs are in an aqueous medium.

In certain embodiments, the methods result in the reduction of side effects associated with the hemolytic properties of a drug and/or excipient of the formulation. In certain embodiments, the method comprises reducing one or more side effects associated with a previously formulated drug by diluting the drug with liposomes before the administration of the drug to a patient. In some embodiments, the patient is a human.

In other embodiments, the compositions and methods provided herein improve the pharmacokinetic and pharmacodynamic properties of a drug.

In one embodiment, provided herein is a method of administering a drug or drugs, said method comprising adding a solution of liposomes to a formulation comprising the drug or drugs.

In one embodiment, provided herein is a method of administering one or more drugs to a subject, the method comprising adding a solution of liposomes to a composition comprising the drug or drugs, and administering the resulting mixture to the subject. In certain embodiments, little to no encapsulation of the one or more drugs by the liposomes occurs in the resulting mixture.

In one embodiment, provided herein is a method of administering a drug, said method comprising adding a solution of liposomes to a formulation comprising the drug, wherein the liposomes are not substantially encapsulated into the liposomes.

In another embodiment, no drug is encapsulated into the liposomes.

In another embodiment, the drug is hydrophobic.

In another embodiment, the drug is not hydrophobic.

In another embodiment, the formulation is lyophilized prior to the addition of the solution of liposomes.

In one embodiment, the formulation is in aqueous medium prior to the addition of the solution of liposomes.

In another embodiment, the method results in at least 50% of the compound being in solution.

In another embodiment, the method results in at least 90% of the compound being in solution.

In one embodiment, provided herein is a method for the delivery of a formulation to a patient, wherein the formulation comprises one or more drugs, the method comprising adding a solution of liposomes to the formulation, wherein the formulation is known to cause side effects in a patient.

In one embodiment, the side effect is an allergic reaction.

In another embodiment, the side effect is hemolysis.

In another embodiment, the drug formulation is aqueous.

In another embodiment, the drug formulation is lyophilized.

In one embodiment, the liposomes comprise one or more phospholipids.

In one embodiment, the phospholipid is phosphatidylcholine.

In another embodiment, the phospholipid is soybean phosphatidylcholine.

In another embodiment, the phospholipid is L-α-phosphatidylcholine.

In another embodiment, the solution of liposomes comprises one or more sugars.

In another embodiment, the sugar is trehalose.

In another embodiment, the resulting solution is 10% by weight trehalose.

In one embodiment, the composition comprises paclitaxel, or a pharmaceutically acceptable salt, hydrate, clathrate or prodrug thereof.

In one embodiment, the composition comprises docetaxel, or a pharmaceutically acceptable salt, hydrate, clathrate or prodrug thereof.

In one embodiment, the composition comprises β-lapachone, or a pharmaceutically acceptable salt, hydrate, clathrate or prodrug thereof.

In one embodiment, the composition comprises transferrin, or a pharmaceutically acceptable salt, hydrate, clathrate or prodrug thereof.

In one embodiment, the composition comprises cyclosporine, or a pharmaceutically acceptable salt, hydrate, clathrate or prodrug thereof.

In another embodiment, the composition is a lyophilized composition further comprising one or more lipids.

In one embodiment, provided herein is a kit comprising:
(a) a single unit dosage form of one or more drugs; and
(b) a solution of liposomes.

In another embodiment, provided herein is a kit comprising:
(a) a single unit dosage form of one or more drugs;
(b) a sealed container comprising a solution of liposomes; and
(c) a device used to administer the one or more drugs in combination with the solution of liposomes.

In another embodiment, the solution of liposomes is an aqueous solution.

In another embodiment, the single unit dosage form is in liquid form.

In another embodiment, the single unit dosage form is an aqueous solution.

In another embodiment, the single unit dosage form is lyophilized.

In one embodiment, provided herein is a method for the delivery of a parenteral drug formulation to a patient, wherein the formulation comprises one or more drugs, the method comprising adding a solution of liposomes to the formulation, wherein the formulation is known to cause side effects in a patient.

In another embodiment, the patient is a human.

In another embodiment, the side effect is an allergic reaction.

In another embodiment, the side effect is hemolysis.

Also provided herein is a pharmaceutical composition prepared by a process comprising adding a solution of liposomes to a composition comprising one or more drugs.

In one embodiment, little to no encapsulation of the one or more drugs by the liposomes occurs in the resulting mixture.

In one embodiment, a method is provided herein for treating a disease or disorder by administering to a patient suffering from said disease or disorder a composition prepared by a process comprising adding a solution of liposomes to a composition comprising one or more drugs.

In another embodiment, little to no encapsulation of the one or more drugs by the liposomes occurs in the resulting mixture.

In one embodiment, the active agent is docetaxel, or a pharmaceutically acceptable salt, hydrate, clathrate or prodrug thereof.

In another embodiment, the disease is a cancer.

In certain embodiments, the solution of liposomes (i.e., liposomal solution used as a diluent) is added to the formulation comprising one or more drugs, in a volume-to-volume (“v/v”) ratio of liposomal solution to drug formulation of, e.g., 0.01:1, 0.1:1, 0.5:1, 1:1, 1:1.5. 1:2, 1:3, 1:4, 1:5, 1:10, 1:20, 1:25, 1:50, 1:100 and 1:1,000. Alternatively, in other embodiments, the solution of liposomes is added to the formulation comprising one or more drugs, in a weight-to-weight (“w/w”) ratio of liposomal solution to drug formulation of e.g., 0.01:1, 0.1:1, 0.5:1, 1:1, 1:1.5. 1:2, 1:3, 1:4, 1:5, 1:10, 1:20, 1:25, 1:50, 1:100 and 1:1,000.

In some embodiments, the diseases or disorders that may be treated by the methods provided herein include, but is not limited to, ontological disorders, proliferative disorders, central nervous system disorders, autoimmune disorders, and inflammatory diseases or disorders. In other embodiments, the methods are directed to the treatment of bacterial, viral or fungal infections.

Proliferative disorders, e.g. cancer, that may be treated by the methods provided herein include, but are not limited to, neoplasms, tumors (malignant and benign) and metastases, or any disease or disorder characterized by uncontrolled cell growth. The cancer may be a primary or metastatic cancer. Specific examples of cancers that can be prevented, managed, treated or ameliorated in accordance with the methods of the invention include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the following: leukemias (e.g., acute leukemia, acute lymphocytic leukemia), acute myelocytic leukemias (e.g., myeloblastic, promyelocytic, myelomonocytic, monocytic, erythroleukemia leukemias and myelodysplastic syndrome), chronic leukemias (e.g., chronic myelocytic (granulocytic) leukemia, chronic lymphocytic leukemia, hairy cell leukemia), polycythemia vera, lymphomas (e.g., Hodgkin's disease, non-Hodgkin's disease), multiple myelomas (e.g., smoldering multiple myeloma, nonsecretory myeloma, osteosclerotic myeloma, plasma cell leukemia, solitary plasmacytoma and extramedullary plasmacytoma), Waldenstrom's macroglobulinemia, monoclonal gammopathy of undetermined significance, benign monoclonal gammopathy, heavy chain disease, bone and connective tissue sarcomas (e.g., bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma), brain tumors (e.g., glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma), breast cancer (e.g., adenocarcinoma, lobular (small cell) carcinoma, intraductal carcinoma, medullary breast cancer, mucinous breast cancer, tubular breast cancer, papillary breast cancer, Paget's disease, and inflammatory breast cancer), adrenal cancer (e.g., pheochromocytom and adrenocortical carcinoma), thyroid cancer (e.g., papillary or follicular thyroid cancer, medullary thyroid cancer and anaplastic thyroid cancer), pancreatic cancer (e.g., insulinoma, gastrinoma, glucagonoma, vipoma, somatostatin-secreting tumor, and carcinoid or islet cell tumor), pituitary cancers (e.g., Cushing's disease, prolactin-secreting tumor, acromegaly, and diabetes insipius), eye cancers (e.g., ocular melanoma such as iris melanoma, choroidal melanoma, and cilliary body melanoma, and retinoblastoma), vaginal cancers (e.g., squamous cell carcinoma, adenocarcinoma, and melanoma), vulvar cancer (e.g., squamous cell carcinoma, melanoma, adenocarcinoma, basal cell carcinoma, sarcoma, and Paget's disease), cervical cancers (e.g., squamous cell carcinoma, and adenocarcinoma), uterine cancers (e.g., endometrial carcinoma and uterine sarcoma), ovarian cancers (e.g., ovarian epithelial carcinoma, borderline tumor, germ cell tumor, and stromal tumor), esophageal cancers (e.g., squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma), stomach cancers (e.g., adenocarcinoma, fungating (polypoid), ulcerating, superficial spreading, diffusely spreading, malignant lymphoma, liposarcoma, fibrosarcoma, and carcinosarcoma), colon cancers; rectal cancers; liver cancers (e.g., hepatocellular carcinoma and hepatoblastoma, gallbladder cancers such as adenocarcinoma), cholangiocarcinomas (e.g., pappillary, nodular, and diffuse), lung cancers (e.g., non-small cell lung cancer, squamous cell carcinoma (epidermoid carcinoma), adenocarcinoma, large-cell carcinoma and small-cell lung cancer), testicular cancers (e.g., germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma), penile cancers, oral cancers (e.g., squamous cell carcinoma), basal cancers, salivary gland cancers (e.g., adenocarcinoma, mucoepidermoid carcinoma, and adenoidcystic carcinoma), pharynx cancers (e.g., squamous cell cancer, and verrucous), skin cancers (e.g., basal cell carcinoma, squamous cell carcinoma and melanoma, superficial spreading melanoma, nodular melanoma, lentigo malignant melanoma, acral lentiginous melanoma), kidney cancers (e.g., renal cell cancer, adenocarcinoma, hypernephroma, fibrosarcoma, transitional cell cancer (renal pelvis and/or uterer)), Wilms' tumor, bladder cancers (e.g., transitional cell carcinoma, squamous cell cancer, adenocarcinoma, carcinosarcoma), myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas, follicular lymphomas, carcinomas with p53 mutations, hormone dependent tumors of the breast, prostate and ovary, precancerous lesions such as familial adenomatous polyposis, and myelodysplastic syndromes.

Other specific diseases and disorders that may be treated by the methods provided herein include, but are not limited to, allergic disorders, inflammation, asthma, arthritis, encephalitis, rheumatoid arthritis, osteoarthritis, psoriatic arthritis, inflammatory osteolysis, chronic or acute obstructive pulmonary disease, chronic or acute pulmonary inflammatory disease, inflammatory bowel disease, Crohn's Disease, gout, Bechet's Disease, Henoch-Schonlein purpura (“HSP”), septic shock, sepsis, meningitis, colitis, inflammation due to reperfusion, psoriasis, fibrosis including pulmonary fibrosis, Parkinson's disease, bradykinesia, muscle rigidity, Parkinsonian tremor, Parkinsonian gait, motion freezing, depression; defective long-term memory, Rubinstein-Taybi syndrome (RTS), dementia, sleep disorders, insomnia, postural instability, hypokinetic disorders, hyperkinetic disorders, synuclein disorders, multiple system atrophies, striatonigral degeneration, olivopontocerebellar atrophy, Shy-Drager syndrome, motor neuron disease with parkinsonian features, Lewy body dementia, Tau pathology disorders, progressive supranculear palsy, corticobasal degeneration, frontotemporal dementia; amyloid pathology disorders, mild cognitive impairment, Alzheimer disease, Alzheimer disease with parkinsonism, Wilson disease, Hallervorden-Spatz disease, Chediak-Hagashi disease, SCA-3 spinocerebellar ataxia, X-linked dystonia parkinsonism, Huntington disease, prion disease, chorea, ballismus, dystonia tremors, Amyotrophic Lateral Sclerosis (“ALS”), CNS trauma, myoclonus, and diseases or disorders associated with undesired immune reaction (e.g., organ rejection associated with an organ transplant).

Viral infections that may be treated by the methods provided herein include, but are not limited to, human immunodeficiency virus (“HIV”), herpes simplex virus type 1, herpes simplex virus type 2, influenza viruses, influenza virus type A, influenze virus type B, parainfluenza virus, human papillomavirus (“HPV”), adenoviruses, rhinoviruses, hepatitis A virus, hepatitis B virus, hepatitis C virus, hepatitis D virus, hepatitis E virus, dengue fever, yellow fever, West Nile virus, Japanese encephalitis virus, GB virus A, GB virus-B, GB virus-C, bovine viral diarrhea virus (“BVDV”), classical swine fever virus (i.e., hog cholera virus), border disease virus, varicella zoster virus, smallpox, measles, rabies virus, arbovirus, cytomegalovirus, mumps virus, poliovirus, coxsackie B virus. Epstein-Barr virus, rubella virus, parvovirus B19, coronaviruses (e.g., SARS coronavirus), astrovirus, norovirus, rotavirus, and adenoviruses.

Fungal infections that may be treated by the methods provided herein include, but are not limited to, aspergillosis, blastomycosis, coccidioidomycosis, cryptococcosis, fungal sinusitis, histoplasmosis, hypersensitivity pneumonitis, mucormycosis, paracoccidioidomycosis, sporotrichosis, and Valley Fever.

Bacterial infections that may be treated by the methods provided herein include, but are not limited to, brucellosis, cholera, leprocy, leptospirosis, shigellosis, trench fever, tularemia, Q fever, Whitmore's disease, yersiniosis, yaws, vibrio vulnificus infections, streptococcus infections, staphylococcus infections and E. coli infections.

In certain embodiments, the methods provided herein result in stable solutions, pharmaceutical compositions, or formulations comprising liposomes and one or more active agents. In these embodiments, the active agent or active agents, remain solubilized for a specified amount of time and do not significantly degrade, aggregate or become otherwise modified (e.g., as determined, for example by HPLC).

In some embodiments, about 70 percent or greater, about 80 percent or greater, or about 90 percent or greater of the active agent remains solubilized after a week after dilution with an acceptable diluent at an elevated temperature (e.g., about 35° C. or higher).

In other embodiments, about 70 percent or greater, about 80 percent or greater, or about 90 percent or greater of the active agent remains solubilized after a week after dilution with an acceptable diluent at room temperature.

In other embodiments, about 70 percent or greater, about 80 percent or greater, or about 90 percent or greater of the active agent remains solubilized after a week at a reduced temperature (e.g., about 10° C. or lower).

In certain embodiments, the methods herein provide formulations that possess increased stability as compared to the conventionally known formulations of the same compound or drug.

Stable formulations provided herein are formulations in which the compounds or drugs of the formulation remain solubilized for a specified amount of time and do not significantly degrade or aggregate or become otherwise modified. In certain embodiments, compounds or drugs of the formulation remain solubilized in the medium (for example, aqueous or organic medium) for a period longer than when the composition is prepared using other methods known in the art. The stability of a formulation can be determined using any well-known methods in the art. One example of such methods is to examine the composition visually. Presumably, when the composition remains clear after a determined period of time, as opposed to becoming turbid, the active ingredient of the composition remains solubilized in the medium. Alternatively, the amount of the active ingredient remaining solubilized after a period of time can be determined using any analytical methods that can provide such information, for example, by HPLC. In some embodiments, greater than about 70 percent, about 80 percent, about 90 percent, about 95 percent or even about 99 percent of the compound or drug of the formulation remains solubilized for at least one week at room temperature after dilution with a diluent, including, but not limited to, the liposomal solutions provided herein.

Furthermore, the formulations provided herein minimize the need for solvents such as CREMOPHOR® EL, N-methylpyrrolidone, dimethylformamide and DMSO in the formulation. Furthermore, in certain embodiments, the need for co-solvents such as ethanol or PEG; co-detergents or surfactants such as Polysorbates or vitamin E; oils such as castor oil or corn oil; proteins such as HSA; and other toxic substances are also minimized using the methods and formulations provided herein.

Pharmaceutical compositions provided herein include single unit dosage forms suitable for oral, topical, mucosal (e.g., nasal, pulmonary, sublingual, vaginal, buccal, or rectal), parenteral (e.g., subcutaneous, intravenous, bolus injection, intramuscular, or intraarterial), or transdermal administration to a patient. Non-limiting examples of dosage forms include: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms provided herein will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms provided herein will vary from one another and will be readily apparent to those skilled in the art. See, e.g., Remington's Pharmaceutical Sciences, 18th ed., Mack Publishing, Easton Pa. (1990).

Suitable excipients, carriers or diluents may be added to or used in the pharmaceutical compositions provided herein. Suitable excipients, carriers or diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The compositions can additionally include lubricating agents, wetting agents, emulsifying and suspending agents, preserving agents, sweetening agents or flavoring agents. In certain embodiments, the compositions provided herein may be formulated so as to provide quick, sustained or delayed response of the active ingredient after administration to the patient. Disintegrants or lubricants may also be used in pharmaceutical compositions and dosage forms provided herein.

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, lyophilized or dry products ready to be dissolved or suspended in a pharmaceutically acceptable vehicle for injection, suspensions ready for injection, and emulsions.

Suitable vehicles that can be used to provide the parenteral dosage forms provided herein are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; and water-miscible vehicles such as, but not limited to, glycerol, ethyl alcohol, polyethylene glycol, and polypropylene glycol. Compounds that further increase the solubility of one or more of the active ingredients disclosed herein can also be incorporated into the parenteral dosage forms provided herein.

Depending on the specific tissue to be treated, additional components may be used prior to, in conjunction with, or subsequent to treatment with the formulations provided herein. For example, additional penetration enhancers can be used to assist in delivering the active ingredients to the tissue. The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

Compositions provided herein may be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein can be readily selected for use with the compositions provided herein.

In some cases, formulations and/or compositions provided herein are not administered to a patient at the same time or by the same route of administration, but instead are provided as kits which, when used by the medical practitioner, can simplify the administration of appropriate amounts of active ingredients to a patient. In one embodiment, the kit comprises a single unit dosage form of a formulation or composition provided herein, and a single unit dosage form of another agent that may be used in combination with the formulations or compositions provided herein. In another embodiment, the kit comprises a single unit dosage form of a formulation or composition provided herein, and a Kits may further comprise devices that are used to administer the active ingredients. Examples of such devices include, but are not limited to, syringes, drip bags, patches, and inhalers.

The kits provided herein may further comprise pharmaceutically acceptable vehicles that can be used to administer one or more active ingredients. For example, if an active ingredient is provided in a solid form (e.g., lyophilized form) that must be reconstituted for parenteral administration, the kit may comprise a sealed container of a suitable vehicle in which the lyophilized compound can be dissolved to form a particulate-free sterile solution that is suitable for parenteral administration. In one embodiment, the pharmaceutically acceptable vehicle comprises liposomes. In another embodiment, the pharmaceutically acceptable vehicle is an aqueous liposomal solution.

All references cited herein are incorporated herein by reference in their entireties and for all purposes to the same extent as if each individual publication, patent or patent application was specifically and individually indicated to be incorporated by reference in its entirety for all purposes.

The methods and compositions provided herein are demonstrated by reference to the following non-limiting examples. It will be apparent to those skilled in the art that many modifications, both to materials and methods, can be practiced without departing from the spirit and scope of methods and compositions provided herein.

### EXAMPLES

### Example 1

### Paclitaxel Formulation Using Miracuxol™

A Miracuxol™ preparation was prepared by combining 50 mg of paclitaxel with 10 mL of an aqueous 100 mM sodium oleate solution, and lyophilizing the resulting mixture.

Lyophilized Miracuxol™ was hemolytic when diluted in isotonic sugars (e.g., 5% w/w D5W, 10% w/w sucrose or 10% w/w trehalose), 4% w/w solutol, 5% w/w cremophor or 2.5% w/w glycerol. Lyophilized Miracuxol™ was not hemolytic when the product was diluted with isotonic 6% w/w liposome containing 6% w/w soybean phosphatidyl choline and 10% w/w trehalose.

### Example 2

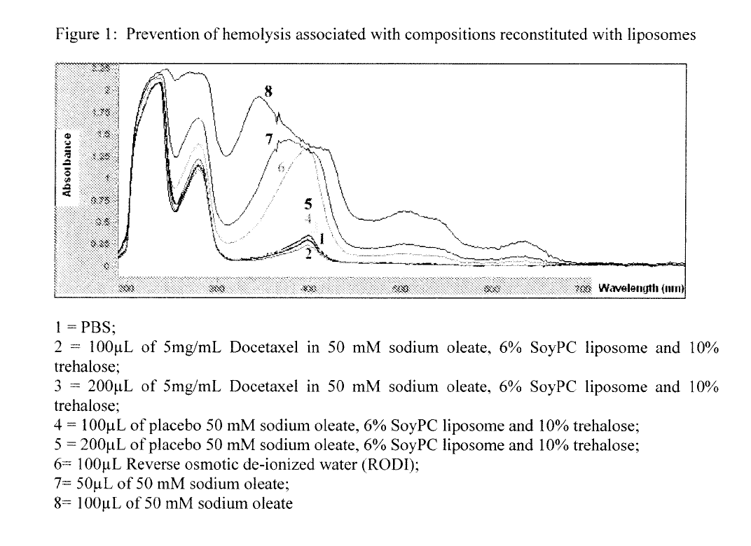

### Reduction of Hemolysis Associated with Compositions Reconstituted with Liposomes (FIG. 1)

100 μL of a 5 mg/mL lyophilized docetaxel preparation (50 mM sodium oleate reconstituted with 6% liposome (Doc-100), 200 μL of 5 mg/mL lyophilized docetaxel containing 50 mM sodium oleate and reconstituted with 6% liposome (Doc-200), 100 mL of placebo containing 50 mM sodium oleate and reconstituted with 6% liposome (Placebo-100), 200 μL of placebo containing 50 mM sodium oleate and reconstituted with 6% liposome (Placebo-200), 200 mL of Reverse osmotic deionized water (RODI (positive control)), 50 n1, of 50 mM sodium oleate (Na oleate-50) in isotonic 2.5% glycerol or 100 μL of 50 mM sodium oleate (Na oleate-100), or 200 μL of phosphate buffered saline (negative control)) was added to 1.4 mL of bovine blood treated with heparin. Doc-100 and Doc-200 were prepared using procedures similar to those in Examples 1 and 2. The samples were incubated at room temperature for 10 minutes, and then centrifuged at 8000 rpm for 20 minutes. The supernatant was used for spectral analysis. The absorbance of the samples was measured by UV-scanning the samples at wavelengths from 190 nm to 810 nm. The high absorbance at 398 nm is due to hemolysis of the blood. As shown in FIG. 1, Doc-100 and Doc-200 caused no hemolysis similar to PBS control. RODI, Na oleate-50 or Na oleate-100 without liposome caused complete hemolysis.

### Example 3

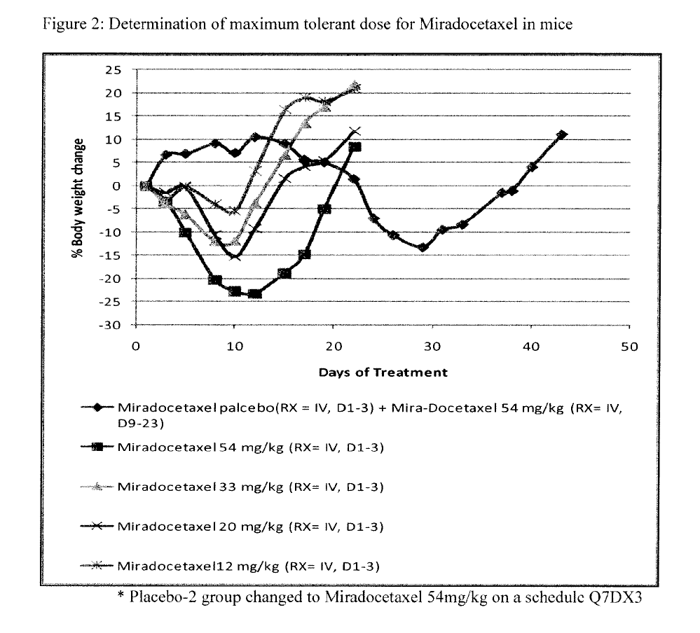

### Determination of Maximum Tolerant Dose of Miradocetaxel™ (FIG. 2)

6-7 week old male athymic mice (nu/nu) were injected with various doses of Miradocetaxel (lyophilized) using 6% liposome as diluents and their percent weight loss was observed. The injections were made every day for 3 days (Q1DX3). A percent weight loss of less than 20% is considered nontoxic. A maximum tolerable dose of 33 mg/kg was observed for Miradocetaxel™. In contrast, the maximum tolerable dose of taxotere formulated in Tween 80-ethanol-saline was found to be 15 mg/kg (data not shown).

What is claimed is:

1. A method of administering one or more drugs to a subject, the method comprising adding a solution of liposomes to a composition comprising the drug or drugs, and administering the resulting mixture to the subject.

2. The method of claim 1, wherein little to no encapsulation of the one or more drugs by the liposomes occurs in the resulting mixture.

3. The method of claim 1, wherein the solution of liposomes is an aqueous solution.

4. The method of claim 1, wherein the liposomes comprise one or more phospholipids.

5. The method of claim 4, wherein the phospholipid is phosphatidyl choline.

6. The method of claim 5, wherein the phospholipid is soybean phosphatidyl choline.

7. The method of claim 1, wherein the composition is a liquid composition.

8. The method of claim 1, wherein the composition is an aqueous solution.

9. The method of claim 1, wherein the composition is lyophilized.

10. The method of claim 3, wherein the solution of liposomes comprises one or more sugars.

11. The method of claim 3, wherein the sugar is trehalose.

12. The method of claim 11, wherein the resulting solution comprises about 10% by weight trehalose.

13. The method of claim 6, wherein the resulting solution comprises about 4% by weight soybean phosphatidyl choline and about 10% by weight trehalose.

14. The method of claim 1, wherein the composition comprises paclitaxel, docetaxel, lapachone, transferrin, cyclosporine, or a pharmaceutically acceptable salt, hydrate, clathrate or prodrug thereof.

15. The method of claim 1, wherein the composition is a lyophilized composition further comprising one or more lipids.

16. A kit comprising:
(a) a single unit dosage form of one or more drugs; and
(b) a solution of liposomes.

17. The kit of claim 16, wherein the solution of liposomes is an aqueous solution.

18. The kit of claim 16, wherein the single unit dosage form is in liquid form.

19. The kit of claim 16, wherein the single unit dosage form is an aqueous solution.

20. The kit of claim 16, wherein the single unit dosage form is lyophilized.

21. A method for the delivery of a parenteral drug to a patient comprising administering to the patient a formulation comprising one or more drugs, the method comprising adding a solution of liposomes to the formulation, wherein the method reduces or alleviates side effects known to be associated with conventional formulations containing said drugs.

22. The method of claim 21, wherein the patient is a human.

23. The method of claim 21, wherein the side effect is an allergic reaction.

24. The method of claim 21, wherein the side effect is hemolysis.

25. A pharmaceutical composition prepared by a process comprising adding a solution of liposomes to a composition comprising one or more drugs under conditions where little or no encapsulation of said drugs occurs.

26. A method of treating cancer comprising administering to a patient a composition of claim 25, wherein the drug in said composition is docetaxel or paclitaxel.
